(19) United States
(12) Patent Application Publication
(10) Pub. No.: US 2026/0248380 A1
(43) Pub. Date: Aug. 27, 2026

(54) INFORMATION PROCESSING APPARATUS AND PROGRAM

(71) Applicant: Makoto KOYAMA, Chiba (JP)

(72) Inventor: Makoto KOYAMA, Chiba (JP)

(21) Appl. No.: 19/332,081

(22) Filed: Sep. 18, 2025

(30) Foreign Application Priority Data
Feb. 26, 2025 (JP) 2025-029560

(51) Int. Cl. A61B 3/024; A61B 3/00; G06T 7/00

(52) U.S. Cl. CPC A61B 3/024; A61B 3/0025; A61B 3/0058; G06T 7/0016; G06T 2207/10101; G06T 2207/20081; G06T 2207/30041

(57) ABSTRACT

An information processing apparatus capable of displaying necessary information in an arranged manner when the results of the visual field examination are referred to, and a program therefor are provided. An information processing device that acquires first estimated visual field information within the central 30 degrees or 24 degrees of the left and right eyes, and second estimated visual field information within the central 10 degrees of the left and right eyes, at a specified date and time based on examination results of a subject. Based on this first and second estimated visual field information, the device synthesizes the estimated visual field of the subject's left and right eyes at the specified date and time, and displays a pair of visual field images arranged to represent the respective estimated visual fields.

### CROSS-REFERENCE TO RELATED APPLICATIONS

The present application claims the priority to and benefit of Japanese Patent Application No. 2025-029560 filed on Feb. 26, 2025. The Japanese Patent Application No. 2025-029560 filed on Feb. 26, 2025 is incorporated by reference herein in its entirety.

### BACKGROUND OF THE INVENTION

### Field of the Invention

The present disclosure relates to an information processing apparatus and a program for processing information relating to the visual field of a person undergoing a medical examination.

### Brief Description of the Related Art

A visual field examination is important for determining a treatment method for glaucoma, and the like, but a result of the examination is shown by presenting the measurement results relating to visions at a plurality of measurement points within the visual field of the right eye or the left eye, and the weighted mean deviation (MD) from the vision of a normal person of the same age as the person undergoing the examination

Here, in general, there are large variations in values of the measurement results at respective measurement points (value of the visible brightness, etc.), even in the case of comparison between the values at the adjacent measurement points. Thus, when the visual field information is displayed by a grayscale image, the value of the measurement result is quantized, and thereafter, made into a grayscale image. Therefore, although an overview can be grasped, detailed information cannot be obtained. Thus, currently, numerical values of the measurement results are presented together with the grayscale image.

Non-Patent Document 1: inet: Surabhi Ruia, Koushik Tripathy, “Humphrey Visual Field” [online], 2023.8.25, National Library of Medicine, [retrieved on January 31, 2025], Internet <URL:https://www.ncbi.nlm.nih.gov/books/NBK585112/>

In order to make a diagnosis on the basis of a result of a visual field examination, a doctor would like to refer to a large amount of information such as:
grayscale image,
numerical value display,
p-value of total deviation,
p-value of pattern deviation,
   regarding each of the 24-degree (or 30-degree) visual field and the 10-degree visual field, with respect to each of the right-eye and the left-eye of the person undergoing the examination.

However, in the above-mentioned conventional information processing apparatus for processing the information of the visual field examination results, information of the 24-degree visual field (or 30-degree visual field, hereinbelow, simply referred to as 24-degree visual field which can be replaced by 30-degree visual field) and information of the 10-degree visual field are separately displayed, and thus, the display should be switched many times and comparing the information is not possible.

Further, since the visual field examination takes time, and imposes a large burden on a person undergoing the examination, usually, the examination for obtaining the 24-degree visual field, and the examination for obtaining the 10-degree visual field are not performed on the same day. Therefore, even if referring to the information by switching is acceptable, there remains a problem that the examination results of the different days are referred to.

Recently, there has been an example that a grayscale image or a number display representing the result of the 10-degree visual field examination is synthesized and displayed on a part (the part corresponding to the result of the 10-degree visual field examination) of a grayscale image or a number display representing the result of the 24-degree visual field examination. However, due to the above-mentioned circumstances, the synthesized information are the examination results of the different days, and thus the displayed results can merely be information for reference.

### SUMMARY OF THE INVENTION

The present disclosure has been considered in view of the above drawbacks, and one of the objectives of the present disclosure is to provide an information processing apparatus capable of displaying necessary information in an arranged manner when the results of the visual field examination are referred to, and a program therefor.

In order to solve the above-mentioned drawbacks of the prior arts, one aspect of the present disclosure is an information processing apparatus comprising: an acquisition device which acquires first estimated visual field information and second estimated visual field information of a person undergoing a medical examination obtained on the basis of an examination result of the person undergoing the medical examination, the first estimated visual field information being estimated visual field information within central 30-degree or 24-degree of the left-eye and the right-eye at a designated date and time, and the second estimated visual field information being estimated visual field information within central 10-degree of the left-eye and the right-eye at the designated date and time; a synthesis device which synthesizes an estimated visual field of the left-eye and an estimated visual field of the right-eye, at the designated date and time, with respect to the person undergoing the medical examination, on the basis of the first estimated visual field information and the second estimated visual field information; and a display device which generates a pair of visual field images respectively representing the estimated visual field of the left-eye and the estimated visual field of the right-eye, at the designated date and time, with respect to the person undergoing the medical examination, and displays the visual field image of the left-eye and the visual field image of the right-eye, in an arranged manner.

According to the present disclosure, on the basis of the results of a visual field examination, visual field information of a designated date and time, regarding a person undergoing the examination, can be displayed in an arranged manner.

### BRIED DESCRIPTION OF DRAWINGS

FIG. 1 is a configurational block diagram of an information processing apparatus according to an aspect of the present disclosure.

FIG. 2 is a functional block diagram of an information processing apparatus according to an aspect of the present disclosure.

FIG. 3 is an explanatory view showing a configuration example of visual field information to be processed by an information processing apparatus according to an aspect of the present disclosure.

FIG. 4 is an explanatory view showing an example of display by an information processing apparatus according to an aspect of the present disclosure.

FIG. 5 is a flowchart showing an operational example of an information processing apparatus according to an aspect of the present disclosure.

### ASPECTS OF DISCLOSURE

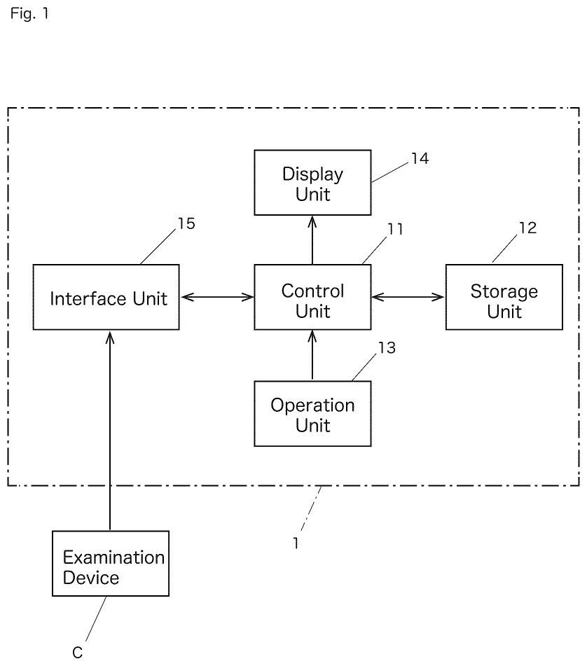

Aspects of the present disclosure will be explained with reference to the drawings. As exemplified in FIG. 1, an example of an information processing apparatus 1 according to an aspect of the present disclosure can be realized by an ordinary computer which comprises a control unit 11, a storage unit 12, an operation unit 13, a display unit 14, and an interface unit 15.

In the present aspect, the control unit 11 is a program-controlled device such as a CPU, which operates in accordance with a program stored in the storage unit 12. According to the present aspect, the control unit 11 obtains an examination result of a person undergoing the medical examination through, for example, an interface unit 15. The control unit 11 obtains first estimated visual field information and second estimated visual field information of the relevant person on the basis of the examination result, the first estimated visual field information being visual field information within the central 30-degree or 24-degree of the left-eye and the right-eye on a designated date and time, and the second estimated visual field information being visual field information within the central 10-degree of the left-eye and the right-eye on the designated date and time; and synthesizes an estimated visual field of the left-eye and the right-eye of the relevant person on the designated date and time, on the basis of the first estimated visual field information and the second estimated visual field information.

Then, the control unit 11 generates a pair of visual field images, the images representing the estimated visual fields of the left-eye and the right-eye, respectively, of the relevant person, on the designated date and time, and displays the visual field image of the left-eye and the right-eye by arranging the same. An operational example of the control unit 11 will be described below.

The storage unit 12 is a memory device, a disk device, etc., which retains a program to be executed by the control unit 11. Also, the storage unit 12 operates as a work memory of the control unit 11. Further, according to an example of the present aspect, the storage unit 12 stores, in advance, sensitivity threshold information regarding a measurement result of a normal person undergoing the examination (hereinbelow, referred to as normal visual field information), in terms of each attribute such as age, sex, etc., specified by each attribute information (age, sex, etc.) of the person undergoing the examination. Here, the sensitivity threshold information of the normal visual field information represents the sensitivity threshold of the normal person undergoing the examination at each measurement point (measurement point such as 24-2, 10-2 etc.) in the 24-degree visual field (or the 30-degree visual field, hereinbelow, when only the 24-degree visual field is referred to, the 24-degree visual field can be replaced by the 30-degree visual field) and the 10-degree visual field, respectively, the visual field being measured by Humphrey Field Analyzer, and the like.

The operation unit 13 is a mouse, a keyboard, etc., which receives instruction operation from a user, and outputs information representing the content of the received instruction operation to the control unit 11. The display unit 14 is a display, etc., which displays various information such as visual field image, etc., in accordance with the instruction input from the control unit 11.

The interface unit 15 is, for example, a USB (Universal Serial Bus) interface, a network interface, etc., which is connected to an examination device C such as an Optical Coherence Tomography (OCT), and the like., and which receives input of information, etc., representing the visual field examination result output from the examination device C, etc. The interface unit 15 outputs the input information to the control unit 11.

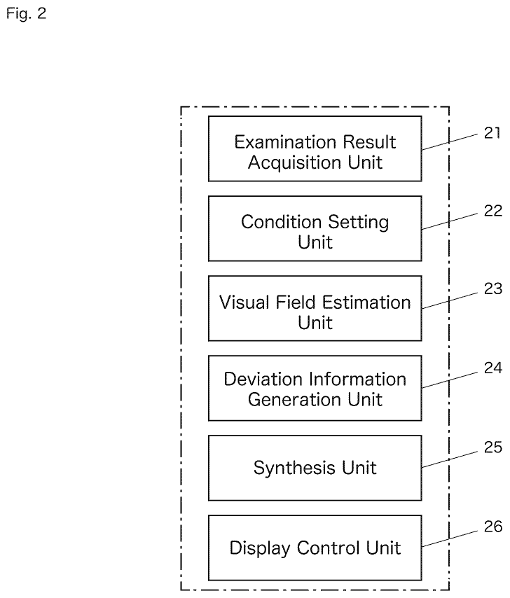

Next, operations of the control unit 11 according to the present aspect will be explained. According to an example of the present aspect, the control unit 11 operates in accordance with the program stored in the storage unit 12, and as exemplified in FIG. 2, realizes a configuration which functionally comprises an examination result acquisition unit 21, a condition setting unit 22, a visual field estimation unit 23, a deviation information generation unit 24, a synthesis unit 25, and a display control unit 26.

The examination result acquisition unit 21 acquires examination result information of a person undergoing a medical examination from the examination device C. Here, the examination result information can be, for example, information output from the Optical Coherence Tomography (OCT), such as image information or three-dimensional structure information of the retinas of the right-eye and the left-eye of the person undergoing the examination.

The condition setting unit 22 sets estimation conditions in the visual field estimation unit 23, and conditions necessary for processing comparison with the normal eye in the deviation information generation unit 24, such as the age, etc., of the person undergoing the examination. Specifically, in accordance with the instructions from the user, the condition setting unit 22 outputs date and time setting which represents the time point of which the visual field is to be estimated, to the visual field estimation unit 23, and outputs age information of the person undergoing the examination, to the deviation information generation unit 24. In the explanation below, the date and time to be output to the visual field estimation unit 23 is referred to as “set date and time”.

Using a machine-trained model having learned the relationship among the image information or the three-dimensional structure information of the retina, the visual field information representing the visual field, and the information representing the change of the visual field, the visual field estimation unit 23 estimates visual field information of the 24-degree visual field on the above-mentioned set date and time regarding the right-eye and the left-eye of the person undergoing the examination, and visual field information of the 10-degree visual field on the above-mentioned set date and time regarding the right-eye and the left-eye of the person undergoing the examination, on the basis of the examination result of the person undergoing the examination acquired by the examination result acquisition unit 21 (the image information or the three-dimensional structure information of the retinas of the right-eye and the left-eye of the person undergoing the examination). Such a machine-trained model can be, for example, a model which has been machine-trained with at least one of the image information and the three-dimensional structure information of the retina regarding a plurality of eyes to be examined and at a plurality of time points, and with visual field relation information relating to the visual field of the eye to be examined at a corresponding time point, to receive input of the image information or the three-dimensional structure information of the retina of each eye to be examined as input information, and to output visual field information and visual field change information estimated on the basis of the input information, using the visual field information representing the visual field obtained based on the visual field relation information at a plurality of time points regarding the corresponding eye to be examined, and the visual field change information representing the change of the visual field. This machine-trained model can be realized by, for example, a model described in Makoto Koyama, et al., “OCT-based Visual Field Estimation Using Segmentation-free 3D CNN Shows Lower Variability than Subjective Standard Automated Perimetry, medRxiv, doi: https://doi.org/10.1101/2024.08.17.24312150.

Here, the visual field information of the 24-degree visual field (corresponding to first estimated visual field information) is estimated information of sensitivity thresholds (can be represented by the minimum brightness value which can be recognized as a position where the bright spot is present by the eye of the person undergoing the examination when the person is gazing at the fixation point) at positions (estimation points) respectively corresponding to 54 measurement points arranged at 6-degree intervals within 24 degrees from the fixation point (central 24-2), used in the Humphrey Field Analyzer, etc. The visual field information of the 10-degree visual field (corresponding to second estimated visual field information) is estimated information of sensitivity thresholds at positions (in the following explanation, referred to as estimation points) respectively corresponding to 68 measurement points arranged at 2-degree intervals within 10 degrees from the fixation point (central 10-2).

The deviation information generation unit 24 compares the visual field information of the 24-degree visual field regarding the right-eye and the left-eye of the person undergoing the examination on the above-mentioned set date and time, and the visual field information of the 10-degree visual field regarding the right-eye and the left-eye of the person undergoing the examination on the above-mentioned set date and time, which have been estimated by the visual field estimation unit 23, with the visual field information of the corresponding normal eye; and generates information representing deviation (usually referred to as total deviation and pattern deviation).

Specifically, the deviation information generation unit 24 acquires visual field information of the right-eye and the left-eye of a normal person corresponding to the attribute information (by way of example, here, the age of the person undergoing the examination) of the person undergoing the examination output from the condition setting unit 22.

The deviation information generation unit 24 calculates the difference of sensitivity thresholds between each visual field information of the right-eye and the left-eye of the person undergoing the examination estimated by the visual field estimation unit 23, and the visual field information of the right-eye and the left-eye of the normal person that has been acquired, at each estimation point and a measurement point corresponding to the estimation point. Thereby, the deviation information generation unit 24 generates information representing the difference of sensitivity thresholds between the normal person and the person undergoing the examination at each measurement point, obtains total deviation and pattern deviation based on the information generated at each measurement point, and calculates statistical information of them such as p-value, etc. For the calculation method of the total deviation, the pattern deviation, and the statistical information of them, widely known methods can be adopted, and thus, detailed description therefor is omitted here.

One of the characteristic features of the example of the present aspect, the visual field estimation unit 23 obtains the visual field information at each estimation point in the 24-degree visual field at a set date and time, as well as the visual field information at each estimation point in the 10-degree visual field at the same date and time, and corresponding thereto, the deviation information generation unit 24 generates the total deviation and the pattern deviation of the 24-degree visual field at the set date and time, and the statistical information of them, as well as the total deviation and the pattern deviation of the 10-degree visual field at the set date and time, and the statistical information of them.

The synthesis unit 25 synthesizes the visual field information estimated with respect to the left-eye of the person undergoing the examination (information representing the estimated visual field of the left-eye of the person undergoing the examination at the set date and time), on the basis of the visual field information of the 24-degree visual field with respect to the left-eye of the person undergoing the examination at the set date and time, and the visual field information of the 10-degree visual field with respect to the left-eye of the person undergoing the examination at the set date and time, which have been obtained by the visual field estimation unit 23. Also, the synthesis unit 25 synthesizes the visual field information estimated with respect to the right-eye of the person undergoing the examination (information representing the estimated visual field of the right-eye of the person undergoing the examination at the set date and time), on the basis of the visual field information of the 24-degree visual field with respect to the right-eye of the person undergoing the examination at the set date and time, and the visual field information of the 10-degree visual field with respect to the right-eye of the person undergoing the examination at the set date and time. Hereinbelow, the visual field information obtained by the synthesis is referred to as the synthesized visual field information.

Further, the synthesis unit 25 generates synthesized total deviation and synthesized pattern deviation with respect to the right-eye and the left-eye, respectively, by synthesizing the information regarding the total deviation and the pattern deviation corresponding to the 24-degree visual field, and the information regarding the total deviation and the pattern deviation corresponding to the 10-degree visual field, which have been generated by the deviation information generation unit 24. These synthesis processes of the synthesized visual field information, the synthesized total deviation, and the synthesized pattern deviation will be described in detail below.

The display control unit 26 outputs information representing the estimated visual field of the left-eye of the person undergoing the examination at the set date and time, and information representing the estimated visual field of the right-eye of the person undergoing the examination at the set date and time, which have been synthesized by the synthesis unit 25, by displaying the same in an arranged manner on the display unit 14.

### Synthesis Process by Synthesis Unit

Here, an example of the synthesis process by the synthesis unit 25 will be explained. The synthesis process by the synthesis unit 25 is performed for the visual field information and the total deviation, for the visual field information and the pattern deviation, or for the visual field information and the total deviation and the pattern deviation. The synthesis process is performed as in the examples described below. Two examples of the synthesis process will be explained below. However, the method for the synthesis process is not limited to these two examples.

### Synthesis Process Example 1

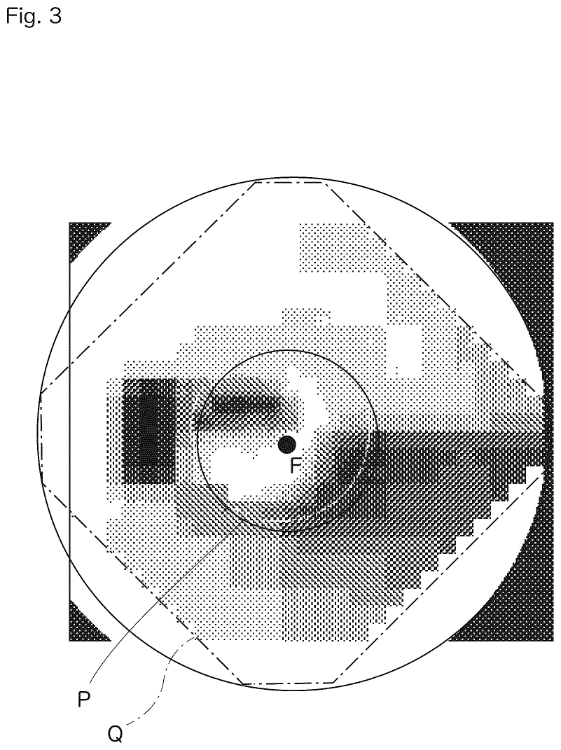

(1) For the values of the sensitivity threshold, the total deviation, and the pattern deviation at the measurement points located in a range of exceeding 10-degree from the center (fixation point) F and within the 24-degree visual field (in FIG. 3, the part within Q and outside of P), the synthesis unit 25 uses the values of the 24-degree visual field themselves as they are.
(2) For the values of the sensitivity threshold, the total deviation, and the pattern deviation at the measurement points in the 10-degree visual field located within 10-degree from the center (in FIG. 3, the part within P), the synthesis unit 25 uses the values of the 10-degree visual field themselves as they are.

By these processes, the synthesis unit 25 determines the sensitivity threshold value, the total deviation value, and the pattern deviation value at each of the measurement points located in a range of exceeding 10-degree from the center and within the 24-degree visual field (the part within Q and outside of P), and determines the sensitivity threshold value, the total deviation value, and the pattern deviation value at each of the measurement points in the 10-degree visual field located within 10-degree from the center (the part within P), respectively.

### Synthesis Process Example 2

Further, the synthesis unit 25 can perform the synthesis process as below. Namely,
(1) For the sensitivity threshold, the total deviation, and the pattern deviation at the measurement points located in a range of exceeding 10-degree from the center and within the 24-degree visual field (in FIG. 3, the part within Q and outside of P), the synthesis unit 25 uses the values of the 24-degree visual field themselves as they are.
(2′) With respect to the measurement points in the 10-degree visual field located within 10-degree from the center (in FIG. 3, within P), the synthesis unit 25 sequentially selects a noted measurement point, and performs following process.

The synthesis unit 25 obtains a sensitivity threshold T(x, y) of a noted estimation point by statistical calculation based on the sensitivity threshold t10(x, y) at a corresponding estimation point with respect to the visual field information of the 10-degree visual field, and the sensitivity threshold t24(xi, yi) (here, i=1, 2, . . . n) of n-pieces of (n being an integer of 1 or more) of estimation points from the closest one to the noted estimation points from among the estimation points with respect to the visual field information of the 24-degree visual field. This calculation can be, for example, a weighted average using the weight determined based on the distance from the noted estimation point.

For example, in FIG. 3, when the estimation point P0 is determined as a noted estimation point from among the estimation points with respect to the visual field information of the 24-degree visual field, for the estimation point at a position closest (n=1) to the noted estimation point P0, and for each of the close n-pieces of estimation points (n-pieces of estimation points selected in the order from the smallest distance from the noted estimation point P0),

$$T ( x , y ) = α \cdot t 10 ( x , y ) + β \cdot \sum t 24 ( xi , yi ) / ( ri \cdot n )$$

is calculated, using the sensitivity threshold t24(xi, yi) (i=1, 2, . . . n) at the relevant estimation point, and the distance (can be Euclidean distance) ri from the position (x, y) of the noted estimation point P0 to the relevant estimation point, with the proviso that each of α, β is a positive real number to satisfy α+β=1, and Σ is i=1, 2, . . . n.

By these processes, as exemplified in FIG. 3, the synthesis unit 25 can respectively determine the sensitivity threshold at the estimation point located in the range of exceeding 10-degree from the center and within the 24-degree visual field (the part within Q and outside of P), and the sensitivity threshold at the estimation point in the 10-degree visual field of withing 10-degree from the center (within P).

Further, in the synthesis process of the total deviation and the pattern deviation, the deviation (total deviation or pattern deviation) value D (x, y) is obtained by statistical calculation based on the deviation value d10 (x, y) at the corresponding measurement point in the 10-degree visual field, and the deviation value d24 (xi, yi) (here, i=1, 2, . . . n) at the n-pieces (n being an integer of 1 or more) of measurement points closest to the position of the noted measurement point among the measurement points in the 24-degree visual field. This calculation can be, for example, a weighted average using the weight determined in accordance with the distance from the noted measurement point.

For example, provided that the measurement point corresponding to the estimation point P0 in FIG. 3 is the noted measurement point, for the measurement point closest (n=1) to the position of the noted measurement point, or each of the close n-neighbor of measurement points (n-pieces of measurement points selected in the order from the smallest distance from the noted measurement point), among the measurement points in the 24-degree visual field, the synthesis unit 25 determines the deviation value by:

$$D ( x , y ) = α \cdot d 10 ( x , y ) + β \cdot \sum d 24 ( xi , yi ) / ( ri \cdot n )$$

using the deviation value d24(xi, yi) (i=1, 2, . . . n) at the relevant measurement point, and the distance (can be Euclidean distance) ri from the position (x, y) of the noted measurement point to the relevant measurement point, with the proviso that each of α, β is a positive real number to satisfy α+β=1, and Σ is i=1, 2, . . . n.

### Display Process by Display Control Unit

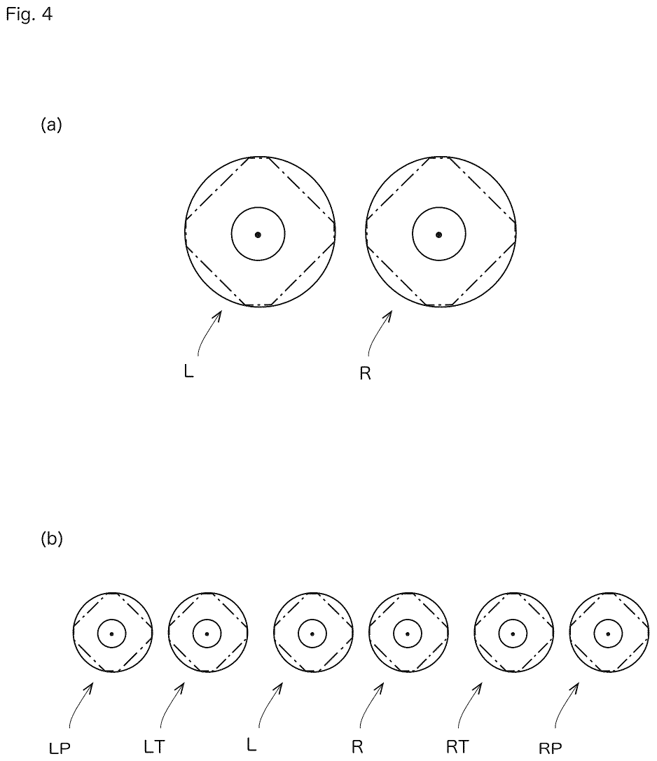

The display control unit 26 displays the synthesized visual field information of the left-eye (left-eye visual field image: L) and the synthesized visual field information of the right-eye (right-eye visual field image: R), which have been synthesized by the synthesis unit 25, by arranging them in a line (side-by-side) as exemplified in FIG. 4(a). In the example of FIG. 4, each of the synthesized visual field information of the right-eye and the synthesized visual field information of the left-eye is displayed as an image which represents the sensitivity threshold at the examination point (which is the display point) included in the visual field information, by grayscale pixels.

Further, the display control unit 26 can display at least one of the synthesized total deviation and the synthesized pattern deviation in the arranged manner, together with the synthesized visual field information of the right-eye and the synthesized visual field information of the left-eye of the person undergoing the examination at the set date and time, which have been synthesized by the synthesis unit 25, displayed in the arranged manner. Each of the synthesized total deviation and the synthesized pattern can also be displayed as an image which represents the value at the examination point (display point) included therein, by grayscale pixels.

As exemplified in FIG. 4(b), the display control unit 26 displays the synthesized pattern deviation of the left-eye (left-eye deviation image: LP), the synthesized total deviation of the left-eye (left-eye deviation image: LT), the synthesized visual field information of the left-eye (left-eye visual field image: L), the synthesized visual field information of the right-eye (right-eye visual field image: R), the synthesized total deviation of the right-eye (right-eye deviation image: RT), and the synthesized pattern deviation of the right-eye (right-eye deviation image: RP), by arranging them in a line (from the left to the right) in this order.

Alternatively, the display control unit 26 can display the synthesized visual field information of the left-eye (left-eye visual field image: L), the synthesized pattern deviation of the left-eye (left-eye deviation image: LP), the synthesized total deviation of the left-eye (left-eye deviation image: LT), the synthesized total deviation of the right-eye (right-eye deviation image: RT), the synthesized pattern deviation of the right-eye (right-eye deviation image: RP), and the synthesized visual field information of the right-eye (right-eye visual field image: R), by arranging them in a line (from the left to the right) in this order. Further, in these arrangements, the order of the synthesized total deviation and the synthesized pattern deviation can be reversed.

Also, the display control unit 26 can change the order of the displayed information in response to the instructions by the user.

### Another Example of Synthesizing Deviation

In the above example, the method for generating the information of the synthesized total deviation or the synthesized pattern deviation in the synthesis unit 25 includes: generating synthesized total deviation and synthesized pattern deviation of the 24-degree visual field and synthesized total deviation and synthesized pattern deviation of the 10-degree visual field, and thereafter, using the value at each estimation point in the 24-degree visual field itself as it is, for each estimation point located in the 24-degree visual field and outside of the 10-degree visual field, and using the value obtained by statistic calculation based on the value at each estimation point in the 24-degree visual field and the value at the estimation point in the 10-degree visual field, for each estimation point within the 10-degree visual field. However, the present aspect is not limited to this example.

The synthesis unit 25 generates the synthesized visual field information, and also performs a synthesis process the visual field information of the 24-degree visual field and the visual field information of the 10-degree visual field of the normal person to generate synthesized visual field information of the 24-degree visual field (referred to as the synthesized normal visual field information).

The synthesis of the visual field information regarding the normal person can be performed by a method same as the synthesis method mentioned above. In this example, the synthesis unit 25 obtains the synthesized total deviation and the synthesized pattern deviation on the basis of the difference between the synthesized visual field information and the synthesized normal visual field information.

### Operations

The information processing apparatus 1 according to the present aspect basically comprises the above-mentioned configuration, and operates as an example below. In the example mentioned below, the storage unit 12 of the information processing apparatus 1 retains, in advance, the normal visual field information for each attribute information (age, sex, etc.). In the explanation herein, the normal visual field information is the one measured by the Humphrey Field Analyzer, etc.

A user (doctor) of this information processing apparatus 1 examines the right-eye and the left-eye of a person undergoing a medical examination, by using the Optical Coherence Tomography (OCT), etc., and obtains the image information or the three-dimensional structure information (examination result) of the retinas of the right-eye and the left-eye of the person undergoing the examination.

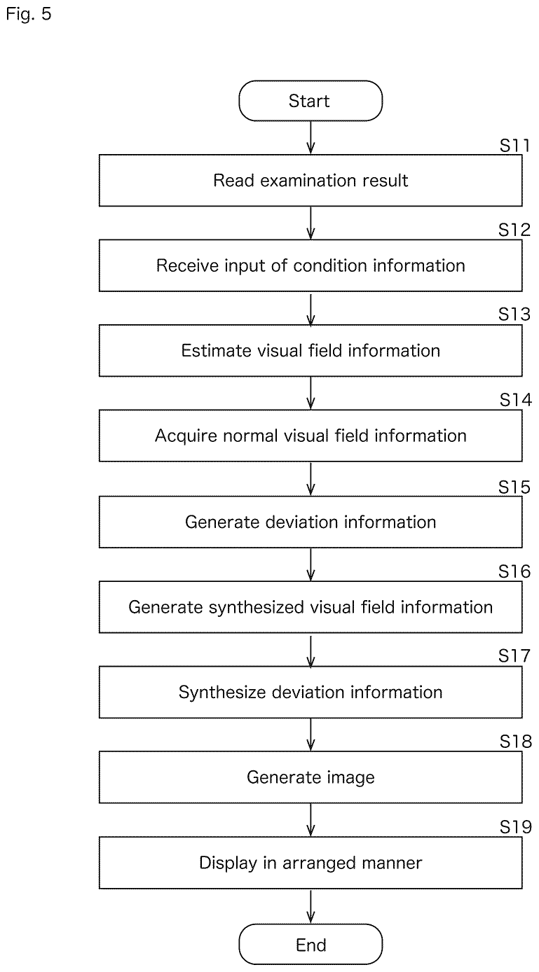

The user instructs the information processing apparatus 1 to initiate processes exemplified in FIG. 5, and to read the examination result (S11). Further, the user inputs the set date and time representing the time point at which visual field information is estimated with respect to the eye of the person undergoing the examination, as condition information (S12).

The information processing apparatus 1 estimates the visual field information of the 24-degree visual field at the set date and time with respect to the right-eye and the left-eye of the person undergoing the examination, and the visual field information of the 10-degree visual field at the set date and time with respect to the right-eye and the left-eye of the person undergoing the examination, on the basis of the examination result read in Step S11 and the information of the set date and time input in Step S12 (S13). As already mentioned above, the estimation process can be performed by a machine-trained model which has learned the relationship among the image information or the three-dimensional structure information of the retina, the visual field information representing the visual field, and the information representing the change of the visual field.

Further, the information processing apparatus 1 reads out the normal visual field information (information of 24-degree visual field and 10-degree visual field) corresponding to the attribute information of the person undergoing the examination (S14). Then, the information processing apparatus 1 compares the visual field information of the 24-degree visual field at the set date and time with respect to the right-eye and the left-eye of the person undergoing the examination, and the visual field information of the 10-degree visual field at the set date and time with respect to the right-eye and the left-eye of the person undergoing the examination, that have been estimated in Step S13, with the normal visual field information that has been read out in Step S14, and generates information representing the deviation (total deviation and pattern deviation)(S15).

On the basis of the visual field information generated in Step S13, the information processing apparatus 1 synthesizes the synthesized visual field information with respect to each of the right-eye and the left-eye of the person undergoing the examination (S16). Further, with respect to the information representing the deviation that has been generated in Step S15, the information processing apparatus 1 generates the synthesized total deviation and the synthesized pattern deviation of the right-eye and the left-eye, respectively, by synthesizing the total deviation information and the pattern deviation information corresponding to the 24-degree visual field, and the total deviation information and the pattern deviation information corresponding to the 10-degree visual field (S17).

The information processing apparatus 1 generates images respectively representing the synthesized visual field information, the synthesized total deviation, and the synthesized pattern deviation with respect to the right-eye and the left-eye of the person undergoing the examination, which have been generated in Step S16 and Step S17 (S18); and displays the left-eye synthesized pattern deviation (left-eye deviation image: LP), the left-eye synthesized total deviation (left-eye deviation image: LT), the left-eye synthesized visual field information (left-eye visual field image: L), the right-eye synthesized visual field information (right-eye visual field image: R), the right-eye synthesized total deviation (right-eye deviation image: RT), and the right-eye synthesized pattern deviation (right-eye deviation image: RP) by arranging them in a line (from the left to the right) in this order (arranged display: S19). The resulting display is the same as the one exemplified in FIG. 4(b) which is already explained above.

Therefore, according to an example of the present aspect, on the basis of the results of the visual field examination, the visual field information of the person undergoing the examination at a designated date and time can be displayed in an arranged manner. Also, according to an example of the present aspect, the estimated visual field based on the examination result by the OCT is used, and thus, compared to the visual field information obtained by actual measurement by the Humphrey Field Analyzer, etc., there is a small variance in the sensitivity thresholds (refer to the above-mentioned paper by Makoto Koyama, et al.). Thus, the sensitivity thresholds at adjacent display points showed a low degree of variability. If, for example, the estimated sensitivity thresholds, the deviation values, and the like, are quantized into 256 levels of grayscale values to generate grayscale images, the generated images are not unnatural.

### Example for Displaying Another Information

The information processing apparatus 1 not only obtains the visual field information and the deviation information explained above, but also calculates statistics obtained from the above information, and other information such as the information estimated by the machine-trained model, and outputs the calculated statistics on the display together with the visual field information and the deviation information, examples of the statistics including:
p-value of the total deviation,
p-value of the pattern deviation,
MD value, or
Time-series information (value based on the examination result and the estimation result in the past, for example, MD slope, etc.).

Further, as the time-series information, the information processing apparatus 1 can calculate the change rate of the sensitivity threshold (progression rate of the disease), the progression probability, and the like, at each estimation point (measurement point), on the basis of the estimation result of the present estimation and the estimation result (or the examination result) in the past, regarding the visual field information at each estimation point (measurement point).

The information processing apparatus 1 can perform numerical value display of the synthesized visual field information. Here, the numerical value display refers to displaying an estimated sensitivity threshold at each corresponding estimation point included in the synthesized visual field information.

Also, if there is actual visual field information (not the estimated result) measured by the Humphrey Field Analyzer, etc., the information processing apparatus 1 can additionally display such information.

Further, in case that such actual visual field information is obtained, the MD value, the MD slope, and other information alternative to the MD slope, such as the VFI (Visual Field Index), the TD (Total Deviation), etc. can be obtained and displayed. These calculations are widely known, and thus, the explanation therefor is omitted here.

Accordingly, the information processing apparatus 1 can display the information with respect to the right-eye and the left-eye of the person undergoing the examination, in an arranged manner as desired, the information including:
image information (grayscale image) of the synthesized visual field information,
image information (grayscale image) of the synthesized total deviation,
image information (grayscale image) of the synthesized pattern deviation,
numerical value information of the synthesized visual field information,
numerical value information of the synthesized total deviation,
numerical value information of the synthesized pattern deviation,
p-value of the total deviation,
p-value of the pattern deviation,
MD value,
time-series information,
visual field information actually measured by visual field analyzer,
MD value and time-series information based on the actual visual field information, as well as information alternative to the above. The user instructs the information processing apparatus 1 to set an arrangement suitable for the medical examination of the person undergoing the examination and easy to see for the user, and to display the information.

As an example, the information processing apparatus 1 can display the images of the visual field information and the images of the deviation information exemplified in FIG. 4(b), and can additionally display the MD slope, a graph showing the time-dependent change of the intraocular pressure, change in eye drop administration in the past, a two-dimensional image of OCT representing the examination result, and the like. According to the present disclosure, examples of the time-dependent change information include the MD slope, the progression rate of the disease at each measurement point, progression probability, time-dependent change of the intraocular pressure, and the change in eye drop administration, and the like.

What is claimed is

1. An information processing apparatus comprising:
an acquisition device which acquires first estimated visual field information and second estimated visual field information of a person undergoing a medical examination obtained on the basis of an examination result of the person undergoing the medical examination, the first estimated visual field information being estimated visual field information within central 30-degree or 24-degree of the left-eye and the right-eye at a designated date and time, and the second estimated visual field information being estimated visual field information within central 10-degree of the left-eye and the right-eye at the designated date and time,
a synthesis device which synthesizes an estimated visual field of the left-eye and an estimated visual field of the right-eye, at the designated date and time, with respect to the person undergoing the medical examination, on the basis of the first estimated visual field information and the second estimated visual field information, and
a display device which generates a pair of visual field images respectively representing the estimated visual field of the left-eye and the estimated visual field of the right-eye, at the designated date and time, with respect to the person undergoing the medical examination, and displays the visual field image of the left-eye and the visual field image of the right-eye, in an arranged manner.

2. An information processing apparatus according to claim 1, wherein
the synthesis device synthesizes information representing a sensitivity threshold, as the estimated visual field, at each display point set within the central 30-degree or 24-degree of the left-eye and the right-eye, at the designated date and time, with respect to the person undergoing the medical examination, and
the display device generates a visual field image by arranging pixels representing the sensitivity threshold at each of the display points.

3. An information processing apparatus according to claim 2, wherein
the synthesis device determines information representing the sensitivity threshold and synthesizes the estimated visual field, at each display point set within the central 30-degree or 24-degree and outside of the central 10-degree of the left-eye and the right-eye, at the designated date and time, with respect to the person undergoing the medical examination, on the basis of the first estimated visual field information; and determines information representing the sensitivity threshold and synthesizes the estimated visual field, at each display point set within the central 10-degree of the left-eye and the right-eye, at the designated date and time, with respect to the person undergoing the medical examination, on the basis of the first estimated visual field information and the second estimated visual field image.

4. An information processing apparatus according to claim 3, wherein
the synthesis device obtains the sensitivity threshold at each display point set within the central 10-degree of the left-eye and the right-eye, at the designated date and time, with respect to the person undergoing the medical examination, by statistical calculation between at least the information within the central 10-degree in the first estimated visual field information, and the second estimated visual field image, and synthesizes the estimated visual field.

5. An information processing apparatus according to claim 1, further comprising an analysis device which generates time-dependent change information regarding the estimated visual field of the person undergoing the medical examination, wherein
the display device displays the time-dependent change information generated by the analysis device, together with the visual field image, in an arranged manner.

6. An information processing apparatus according to claim 1, wherein
the acquisition device further acquires first normal visual field information and second normal visual field information, the first normal visual field information being visual field information within the central 30-degree or 24-degree of the left-eye and the right-eye of a normal person, and the second normal visual field information being visual field information within the central 10-degree of the left-eye and the right-eye of the normal person on the designated date and time, and
the display device generates deviation images, each of which represents a difference from the normal state, for the left-eye and the right-eye of the person undergoing the medical examination, respectively, on the basis of the estimated visual fields of the left-eye and the right-eye of the person undergoing the medical examination at the designated date and time, the first normal visual field information, and the second normal visual field information, and displays the generated deviation images together with the visual field images of the left-eye and the right-eye, in an arranged manner.

7. An information processing apparatus according to claim 6, wherein
with respect to the person undergoing the medical examination at the designated date and time, the display device displays a left-side image and a right-side image by arranging them in a line, the left-side image including the left-eye deviation image and the left-eye visual field image which are adjacently arranged, and the right-side image including the right-eye deviation image and the right-eye visual field image which are adjacently arranged.

8. An information processing apparatus according to claim 6, wherein
with respect to the person undergoing the medical examination at the designated date and time, the display device displays the left-side image and the right-side image by arranging them in a line, the left-side image including the left-eye deviation image and the left-eye visual field image which are adjacently arranged, and the right-side image including the right-eye deviation image and the right-eye visual field image which are adjacently arranged, and arranges the images in a line in the order of the left-eye deviation image, the left-eye visual field image, the right-eye visual field image, and the right-eye deviation image, or in the order of the left-eye visual field image, the left-eye deviation image, the right-eye deviation image, and the right-eye visual field image.

9. An information processing apparatus according to claim 1, wherein
the acquisition device which acquires image information or three-dimensional structure information of the retina as an examination result of the person undergoing a medical examination using a machine-trained model, and acquires, on the basis of the acquired image information or three-dimensional structure information, the first estimated visual field information within the central 30-degree or 24-degree of the left-eye and the right-eye of the person undergoing the medical examination at the designated date and time, and the second estimated visual field information within the central 10-degree of the left-eye and the right-eye of the person undergoing the medical examination at the designated date and time, wherein
the machine-trained model has been trained with at least one of the image information and the three-dimensional structure information of the retina regarding a plurality of eyes to be examined and at a plurality of time points, and with visual field relation information relating to the visual field of the eye to be examined at a corresponding time point, so as to output visual field information and visual field change information estimated on the basis of the image information or the three-dimensional structure information of the retina of each eye to be examined as input information, and, using, as training data, the visual field information representing the visual field obtained based on the visual field relation information at the plurality of time points regarding the corresponding eye to be examined, and the visual field change information representing the change of the visual field.

10. A program which functions a computer as:
an acquisition device which acquires first estimated visual field information and second estimated visual field information of a person undergoing a medical examination obtained on the basis of an examination result of the person undergoing the medical examination, the first estimated visual field information being estimated visual field information within central 30-degree or 24-degree of the left-eye and the right-eye at a designated date and time, and the second estimated visual field information being estimated visual field information within central 10-degree of the left-eye and the right-eye at the designated date and time,
a synthesis device which synthesizes an estimated visual field of the left-eye and an estimated visual field of the right-eye, at the designated date and time, with respect to the person undergoing the medical examination, on the basis of the first estimated visual field information and the second estimated visual field information, and
a display device which generates a pair of visual field images respectively representing the estimated visual field of the left-eye and the estimated visual field of the right-eye, at the designated date and time, with respect to the person undergoing the medical examination, and displays the visual field image of the left-eye and the visual field image of the right-eye, in an arranged manner.
